(19) United States
(12) Patent Application Publication
(10) Pub. No.: US 2016/0089440 A1
(43) Pub. Date: Mar. 31, 2016

(54) POTENCY-EXTENDING AGENT OF BOTULINUM TOXIN TYPE-A PRODUCT

(71) Applicant: Jong Wook HAM, Seoul (KR)

(72) Inventor: Jong Wook HAM, Seoul (KR)

(21) Appl. No.: 14/536,623

(22) Filed: Nov. 8, 2014

(30) Foreign Application Priority Data
Sep. 29, 2014 (KR) 10-2014-0130241

(51) Int. Cl. A61K 47/26; A61K 31/167; A61K 47/02

(52) U.S. Cl. CPC A61K 47/26; A61K 47/02; A61K 31/167

(57) ABSTRACT

A potency-extending agent of a botulinum toxin type-A product includes 1 ml of distilled water, 85 of dextrose, 4.28 mg of sodium chloride, and 10 mg of trehalose.

### CROSS-REFERENCE TO RELATED APPLICATIONS

This patent application claims priority under 35 U.S.C. §119 to Korean Patent Application No. 10-2014-0130241, filed on Sep. 29, 2014, in the Korean Intellectual Property Office, the disclosure of which is incorporated by reference herein in its entirety.

### TECHNICAL FIELD

The present invention relates to an extending agent used for dissolving and diluting a botulinum toxin type-A product, and more specifically, to a novel potency-extending agent produced by mixing dextrose, as a main component, natrium chloride (NaCl), as a first stabilizer, lidocaine, as a pain killer, trehalose or sucrose, as a second stabilizer, with distilled water for injection.

### DISCUSSION OF RELATED ART

The conventional commercially available botulinum toxin, particularly botulinum toxin type-A product, which is obtained by freeze-drying together with a stabilizer such as albumin or gelatin, has been used as a therapeutic agent of various clinical symptoms including histrionic spasm, muscle tone, migraine headache, wrinkles, cerebral palsy, hyperhidrosis, etc. Such conventional powder-type agent needs to be diluted with physiological saline for clinical use. The botulinum toxin type-A product, as diluted with physiological saline, experiences unnecessary protein denaturization by as much as 43%, thus dropping its potency preservation rate down to 57%. In other words, after diluted with physiological saline, the potency loss rate of botulinum toxin type-A product reaches as high as about 43%, resulting, in the potency preservation rate being, not more than 57%. This means that the botulinum toxin type-A product, if diluted with a 0.9% NaCl solution (physiological saline) as in the prior art, remains only at a potency of 51% because the stabilizer contained in the toxin product itself does not work sufficiently. Moreover, the toxin product diluted as per the conventional method is subjected to a steady efficacy degradation, and it should be thus consumed within a week.

As such, the botulinum toxin type-A product (Botox®), as diluted with physiological saline as in the conventional art, shows a potency preservation rate of 57%, so that the actual potency of Botox® 100 units becomes 57 units. Accordingly, the relative potency of the 100 units product should be equal to 175 units in order to remain at 100% even after diluted. For better understanding, the following equation may be established: 57% (conventional potency preservation rate): 100 (units)=100% (potency preservation rate):X (units). According to the above equation, X becomes about 175 units. Accordingly, since use of the same quantity as the physiological saline increases the concentration by 1.75 times, the quantity to be diluted should be increased to 1.75 times.

In view of the above problems of the prior art, the inventor has invented, through continuous and repetitive experiments,

a novel potency-extending agent that allows the botulinum toxin type-A product to be left at a potency preservation rate of 100% without potency loss (i.e., zero potency loss rate) even after diluted, thus leading to a 75% increase in available quantity as compared with the prior art. Further. the toxin product, after diluted with the potency-extending agent according to an embodiment of the present invention, may be stored. and distributed for a long time (12 to 24 months) at a low temperature of 2-8° C. without potency loss.

### PRIOR TECHNICAL DOCUMENTS

### Patent Documents

(Patent Document 0001) Korean Patent Application No. 10-2011-0049424

(Patent Document 0002) Korean Patent Application No. 10-2012-0028189

(Patent: Document 0003) Korean Patent Application No. 10-2008-0049914

### SUMMARY

The present invention has been designed to solve the above problems, and an embodiment of the present invention is to provide a novel potency-extending agent that may prevent the botulinum toxin type-A product from losing its potency even after diluted to leave the potency preservation rate at 100%, leading to a 75% increase in potency or quantity.

Another embodiment of the present invention is to provide a novel potency-extending agent that allows for long-term storage and distribution of botulinum toxin type-A product (12 to 24 months) at a low temperature of 2-8° C. without potency loss even after dilution.

According to an embodiment of the present invention, a potency-extending agent of a botulinum toxin type-A product is produced by mixing 1 ml of distilled water for injection with 84-86 mg of dextrose_{:} as a main component, 4.18-4.38 mg, of sodium chloride, as a first stabilizer, and 9-11 mg of trehalose, as a second stabilizer.

In this case, the potency-extending agent may further include 0.9-1.1 mg of lidocaine, as a pain killer, to mitigate a patient's pain upon injection.

Further, according to an embodiment of the present invention, a potency-extending agent of a botulinum toxin type-A product is produced by mixing 1 ml of distilled water for injection with 84-86 mg of dextrose, as a main component, 4.18-4.38 mg of sodium chloride, as a first stabilizer, and 9-11 mg of sucrose, as a second stabilizer.

In this case, the potency-extending agent may further include 0.9-1.1 mg of lidocaine, as a pain killer, to mitigate a patient's pain upon injection.

Further, according to an embodiment of the present invention, a potency-extending agent of a botulinum toxin type-A product is produced by mixing 1 ml of distilled water for injection with 84 -86 mg of dextrose, as a main component, 4.18-4.38 mg of sodium chloride, as a first stabilizer, and 4.5 -5.5 mg of trehalose and 4.5-5.5 mg of sucrose, as a second stabilizer.

In this case, the potency-extending agent may further include 0.9-1.1 mg of lidocaine. as a pain killer, to mitigate a patient's pain upon injection.

In the conventional method of diluting a botulinum toxin type-A product using physiological saline, the botulinum toxin type-A product shows a potency loss rate of 43% and resultantly a potency preservation rate of 57%. However, when diluted with the novel potency-extending agent according to an embodiment of the present invention, the botulinum toxin type-A product may exhibit zero-potency loss, and thus, its potency preservation rate reaches 100%. Accordingly, the present invention provides significant economic advantages.

The botulinum toxin type-A 100 units product exhibits a potency of 57 units when diluted by the existing method. However, an actual potency of 100 units (i.e., a clinical potency of 175 units) may be achieved for the botulinum toxin type-A 100 units product when the product is diluted with a novel potency-extending agent according, to an embodiment of the present invention. As such, the present invention leads to a 75% increase in potency or quantity of botulinum toxin type-A product actually available, thus offering significant economic advantages.

As compared with the existing dilution method, the dilution method according to an embodiment of the present invention may bring about a 75% increase in quantity and resultantly significant economic advantages.

According to an embodiment of the present invention, the dosage of the (botulinum toxin type-A) protein component may be reduced in ratio from 1.75 to 1, thus decreasing drug resistance and general toxicity.

Further, the botulinum toxin type-A product, when diluted with a novel potency-extending agent according to an embodiment of the present invention, may be long-term preserved (12 to 24 months) without potency loss at a low temperature of 2-8° C.

### DETAILED DESCRIPTION OF EMBODIMENTS

Embodiments of the present invention are described below in detail. However, the present invention is not limited thereto, and various changes ma be made thereto without departing from the spirit or scope of the present invention. As used herein, the singular forms “a,” “an,” and “the” are intended to include the plural forms as well unless the context clearly indicates otherwise.

### First Embodiment

A novel potency-extending agent of a botulinum toxin type-A product was produced by mixing 1 ml of distilled water for injection with 85 mg of dextrose, as a main component, 4.28 mg of sodium chloride, as a first stabilizer, and 10 mg of trehalose, as a second stabilizer.

In this case, 1 mg of lidocaine may be added as a pain killer to mitigate the patient's, pain upon injection.

As a result of a dilution experiment using the novel potency-extending anent thus obtained, the botulinum toxin type-A product exhibited a potency loss rate of 0% and a 75% increase in potency. Further, the botulinum toxin type-A product was able to be stored and distributed for a long time (18 to 24 months) at a low temperature of 2-8° C., and its potency was kept constant.

### Second Embodiment

A novel potency-extending agent of a botulinum toxin type-A product was produced by mixing 1 ml of distilled water for injection with 85 mg of dextrose, as a main component, 4.28 mg of sodium chloride, as a first stabilizer, and 10 mg of sucrose, as a second stabilizer.

In this case, 1 mg of lidocaine may be added as a pain killer to mitigate the patient's pain upon injection.

As a result of a dilution experiment using the novel potency-extending agent thus obtained, the botulinum toxin type-A product exhibited a potency loss rate of 0% and a 75% increase in potency. Further, the botulinum toxin type-A product was able to be stored and distributed for a long time (18 to 24 months) at a low temperature of 2-8° C., and its potency was kept constant.

### Third Embodiment

A novel potency-extending agent of a botulinum toxin type-A product was produced by mixing 1 ml of distilled water for injection with 85 mg of dextrose, as a main component, 4.28 mg of sodium chloride, as a first stabilizer, and 10 mg of sucrose, as a second stabilizer.

In this case, 1 mg of lidocaine may be added as a pain killer to mitigate the patient's pain upon injection.

As a result of a dilution experiment using the novel potency-extending agent thus obtained, the botulinum toxin type-A product exhibited a potency loss rate of 0% and a 75% increase in potency. Further, the botulinum toxin type-A product was able to be stored and distributed for a long time (18 to 24 months) at a low temperature of 2-8° C. and its potency was kept constant.

### Fourth Embodiment

A novel potency-extending agent of a botulinum toxin type-A product was produced by mixing 1 ml of distilled water for injection with 95 mg of dextrose, as a main component.

In this case, 1 ml of lidocaine may be added as a pain killer to mitigate the patient's pain upon injection.

As a result of a dilution experiment using the novel potency-extending agent thus obtained, the botulinum toxin type-A product exhibited a potency loss rate of 0% and a 75% increase in potency. Further, the botulinum toxin type-A product was able to be stored and distributed for a long time (13 to 15 months) at a low temperature of 2-8° C. and its potency was kept constant.

### Fifth Embodiment

The botulinum toxin type-A product (Botox®) diluted with physiological saline according to the prior art and Botox® diluted with a novel potency-extending agent according to an embodiment of the present invention were injected into the thigh muscle of mice. The result of the comparative experiment is shown in the following Table 1:

TABLE 1

| | Botox ® | Botox ® (present |
|---|---|---|
| Potency | (conventional art) | invention) |
| 0.1 units | 0 | 2 |
| 0.2 units | 0 | 14 |
| 0.3 units | 2 | 20 |
| 0.4 units | 18 | — |

In Table 1 above, each experimental population is ten.
The points were calculated as follows:
0: when operated well without change
1: when intermittently reacted by a physical stimulus
2: when not reacted by a physical stimulus
(20 points when none of the ten mice react)

Table 2 below shows the resultant data obtained from an experiment performed under substantially the same conditions given in connection with Table 1 after retained at room temperature for about six months.

TABLE 2

| | Botox ® | Botox ® (present |
|---|---|---|
| Potency | (conventional art) | invention) |
| 0.1 units | 0 | 1 |
| 0.2 units | 0 | 15 |
| 0.3 units | 0 | 20 |
| 0.4 units | 0 | — |

While the inventive concept has been shown and described with reference to exemplary embodiments thereof, it will be apparent to those of ordinary skill in the art that various changes in form and detail may be made thereto without departing from the spirit and scope of the inventive concept as defined by the following claims.

1. A potency-extending agent of a botulinum toxin type-A product, the potency-extending agent produced by mixing 1 ml of distilled water for injection with 84-86 mg of dextrose, as a main component, 4.18-4.38 mg of sodium chloride, as a first stabilizer, and 9-11 mg of trehalose or sucrose, as a second stabilizer.

2. The potency-extending agent of claim 1, wherein the potency-extending agent further includes 0.9-1.1 mg of lidocaine, as a pain killer, to mitigate a patient's pain upon injection.

3. The potency-extending agent of claim 1, wherein the potency-extending agent increases the potency of the botulinum toxin type-A product by 75% without potency loss after diluting the botulinum toxin type-A product and the potency-extending agent allows the botulinum toxin type-A product to have a preservation period of 18 to 24 months without potency loss at a low temperature of 2-8° C. after dilution.

4. A potency-extending agent of a botulinum toxin type-A product, the potency-extending agent produced by mixing 1 ml of distilled water for injection with 84-86 mg of dextrose, as a main component, 4.18-4.38 mg of sodium chloride, as a first stabilizer, and 4.5-5.5 mg of trehalose and 4.5-5.5mg of sucrose, as a second stabilizer.

5. The potency-extending agent of claim 4, wherein the potency-extending agent limber includes 0.9-1.1 mg of lidocaine, as a pain killer, to mitigate a patient's pain upon injection.

6. The potency-extending agent of claim 4, wherein the potency-extending agent increases the potency of the botulinum toxin type-A product by 75% without potency loss after diluting the botulinum toxin type-A product and the potency-extending agent allows the botulinum toxin type-A product to have a preservation period of 18 to 24 months without potency loss at a low temperature of 2-8° C. after dilution.

7. A potency-extending agent of a botulinum toxin type-A product, the potency-extending agent comprises 1 ml of distilled water 85 of dextrose, 4.28 mg of sodium chloride, and 10 mg of trehalose.

8. The potency-extending agent of claim 7, wherein the potency-extending agent increases the potency of the botulinum toxin type-A product by 75% without potency loss after diluting the botulinum toxin type-A product and the potency-extending agent allows the botulinum toxin type-A product to have a preservation period of 12 to 15 months without potency loss at a low temperature of 2-8° C. after dilution.
